(19) United States
(12) Patent Application Publication
(10) Pub. No.: US 2008/0102482 A1
(43) Pub. Date: May 1, 2008

(54) Apparatus for Detecting Drugs in a Beverage

(76) Inventors: Stanley Irwin Grossman, London (GB); James Gordon Campbell, Derbyshire (GB)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/583,512

(22) PCT Filed: Dec. 16, 2004
(86) PCT No.: PCT/GB04/05261

§ 371 Date: Aug. 13, 2007

(30) Foreign Application Priority Data
Dec. 19, 2003 (GB) 0329503.7
Mar. 12, 2004 (GB) 0405646.1

(51) Int. Cl. G01N 33/00

(52) U.S. Cl. 435/7.93

(57) ABSTRACT

A test kit for detecting the presence of one or more drugs in a beverage comprising a support having a plurality of detector strips attached thereto. Drugs to be detected are e.g. benzodiazepines, gamma hydroxy butyrate GHB), Ketamines, Ectasy (MDMA), cocaine barbiturates, opiates, cannabis and flunitrazepam (Rohypnol®).

The present invention relates to apparatus for use in testing the presence of foreign substances in a beverage. More particularly it relates to a test kit for detecting the presence of one or more drugs in a beverage.

For ease of reference in the present application the term “drug” and “drugs” will be used to refer to any material which has been added to a beverage without the consent of the consumer. As will be explained in more detail below, the term will include alcohol, prescription pharmaceuticals, over the counter pharmaceuticals, illegal substances and the like.

It has been known for many years for perpetrators wishing to cause harm to a victim to add a drug to the victim's beverage to alter the victim's behaviour or to incapacitate them totally. For example, alcohol may be added to a non-alcoholic drink or drugs may be added to either an alcoholic or non-alcoholic drink. When the victim has drunk the beverage, their mood may be altered, or they may become totally incapacitated. While the victim is suffering from the effect of the drug the perpetrator may take advantage of the victim. In its mildest form, the victim's behaviour may simply be the source of amusement. Whilst this may be the perpetrator's intent, the action can have more serious consequences since the drug dose is generally unknown, as is the effect of the combination of the drug with alcohol. Further the drug may have deleterious effects on any medical condition that the victim may have or may have an adverse interaction with any other medication that the victim may be taking. Unfortunately, there is usually a more sinister intent such as robbery, rape or even murder.

Drugs currently commonly used for illicit addition to beverages include alcohol, drugs from the benzodiazepine family, gamma hydroxybutyrate, 3,4-methylenedioxy-N-methylamphetamine (also known as MDMA or Ecstasy), ketamines, cannabis and the like. Recent studies suggest that there are at least two hundred drugs available legally or illegally which could be secretly applied to beverages for the purpose of disabling or incapacitating the victim.

Of particular concern at the present time is the use of flunitrazepam which is a potent benzodiazepine drug sold under the trade name Rohypnol. Whilst this drug has legitimate uses, it has become known as the “date rape” drug because of its use as an additive to beverages to cause the victim to become amenable to any suggestion and be unable to remember the situation afterwards. It is therefore commonly used to enable the perpetrator to obtain sex from the victim.

In an attempt to overcome this problem, the manufacturers of Rohypnol have added a blue dye to the prescription tablets to prevent their being added to drinks without the user's knowledge. However, there are still illicit forms of flunitrazepam available which are not coloured.

Drug rape usually goes unchecked since the victims are unable to remember the events or details and therefore are unreliable witnesses in any legal action. Even if the victim can remember some details or have suspicions they are often unwilling to come forward.

It is therefore desirable to provide a testing means which will enable users to test their beverage to establish whether it is safe to drink. The benefits of a testing method include that the user knows that he or she is safe. A further benefit of a testing method is that where the presence of a drug is detected, there is a possibility that the perpetrator maybe identified. Any such device could also be used by authorities to screen drinks at the scene of an alleged offence.

Whilst there are a variety of established technologies available for testing for drugs, these are generally based on laboratory or clinical tests and are therefore not suitable for use by the average consumer. They are also usually time consuming and expensive to run and require the use of clean rooms and for strict protocols to be observed. In addition, many of these systems are not able to function in the presence of an acid or alcohol and therefore have limited use in testing for drugs in beverages. These known tests also generally are only able to test for one drug at a time and require resetting and recalibrating for the next drug to be tested.

Various proposals have been made to provide test apparatus which can be used at the site and time of consumption of the beverage. In U.S. Pat. No. 6,153,147 a beverage analysis device is described in which a portion of liquid from the beverage can be captured and drawn into an analysis chamber where it is subjected to a chemical reagent composition and the results of the colour assay are viewed through a window.

In US 2001/0046710 a test strip, toothpick or cotton swab is described which can be immersed into the beverage and which will exhibit a colour change when gamma hydroxybutyrate is present in the beverage.

In GB 2383130 a test strip is described which is particularly useful for the detection of gamma hydroxybutyrate or methylene dioxymethamphetamine. The test strip may be replaced with a coaster having a plurality of testing means.

WO 03/021254 describes a test apparatus in which one or more solid, chemical colorimetric indicators are embedded in the surface of a porous substrate.

Whilst these arrangements go some way to providing a suitable solution to the desirability of providing a test which can be used at the point of consumption, it is still desirable to provide alternative, and preferably improved, arrangements which preferably enable the user to test for one or more of a variety of drugs and which are preferably cost-effective to manufacture and easy to operate.

Thus according to the present invention there is provided a test kit for detecting the presence of one or more drugs in a beverage comprising a support having a plurality of detector strips attached thereto. The strips are may be releasably attached thereto.

In a preferred arrangement of the present invention the kit will contain detector strips for detecting the presence of at least two, and preferably at least three, different drugs. Thus the kit may include at least one strip for detecting the presence of benzodiazepines and at least one detector strip for testing for gamma hydroxy butyrate. It may also include at least one strip for detecting the presence of alcohol.

In a further arrangement further strips may be included to test for one or more of ketamines, 3,4-methylenedioxy-N-methylamphetamine, cannabis, cocaine, barbiturates, opiates, tricyclic antidepressants, acetaminophen, propoxyphene and phencyclidine.

Whilst each detector strip may be configured to detect for more than one drug, in a preferred arrangement, each strip will test for one drug or one drug type only and will therefore be able to be formed of the optimum material for testing for the particular drug.

In one arrangement the kit will include at least two of each type of drug detector strips.

The detector strip may be of any suitable configuration. It will generally be of elongate configuration but other suitable configurations may be used. Where appropriate the strip may be rectangular or square. The strip will comprise the detection means. The strip may be completely supplied as the detection means or may comprise a bed supporting the detection means. The detection means will include assays or reagents which enable the target drug to be detected. For example, immunoassay, chemical spot, enzyme and the like techniques may be employed. For ease of reference, these will be collectively referred to as the “detection system”. The material from which the detection strip is constructed will depend on the detection system to be used. Where more than one detection system is used on different strips, they may be formed from different materials.

The antibodies used in immunoassay techniques are often expensive and have poor tolerance to acids, alcohol, moisture and temperature extremes. However, immunoassay tests maybe developed which are suitable for use in the present invention.

In one arrangement of the present invention a detector strip maybe present which is suitable for the detection of the presence of benzodiazepines. In one arrangement this may be prepared using an immunoassay technique for example a lateral flow competitive immunoassay or lateral flow direct binding immunoassay. Full details of one example of this immunoassay technique can be found in GB2383130 which is incorporated herein by reference.

In brief, a membrane is applied to the strip onto which is placed a pad of gold conjugate, bound to a first antibody having an affinity to the drug being tested, and a stripe, line or other indication from a second antibody is added which has an affinity for the first antibody. When the material comes into contact with the beverage the liquid will travel along the membrane carrying the gold conjugate-antibody complex with it as it migrates. If a drug is present, it will bind to this complex and block any binding sites on the complex. As the complex passes the location of the second antibody, the gold conjugate, which is magenta in colour, does not bind and passes beyond the line, so that no colour change is observed. The failure to produce a colour change would notify the user that there was a drug present.

If no drug is present, the gold conjugate-antibody complex does not have its binding sites blocked and as it passes the stripe of the second antibody binding occurs and a coloured stripe, line or other indication is obtained.

In another embodiment the production of binding to give a visual signal may be obtained by means of a sandwich assay instead of lateral flow technology. In this embodiment the constituents already described are placed through a porous membrane and the reaction takes place through the sandwich, revealing the result as a spot, line or other indication in the absence of a drug in the benzodiazepines group. The technology may function either as a competitive sandwich immunoassay or as a direct sandwich immunoassay.

Examples of suitable “other indications” include a tick or a cross or a suitable word such as “pass” or “OK”.

Immunoassay techniques may also be utilized to provide detector strips which may be used to detect for the presence of ketamines, 3,4-methylenedioxy-N-methylamphetamine, cocaine, barbiturates, opiates, tricyclic antidepressants, acetaminophen, propoxyphene and phencyclidine.

Whilst the detector strip for testing for benzodiazepines may be prepared using immunodiagnostic technology, other techniques for example colour change chemistry, may be used.

In the detector strips of the present invention, chemical spot techniques may be used as the detection means. Chemical spot tests rely on a reaction, such as a redox reaction, between the substance being tested and the chemical used to perform the reaction which changes colour either because it produces a complex or because there is an alkalinity/acidity/pH change. Usually there is a colour change when the suspect drug is present. In one arrangement of the present invention the detector strip for detecting gamma hydroxybutyrate utilises chemical spot techniques.

In one embodiment of the present invention the test for gamma hydroxybutyrate operates by applying a small spot of chemical indicator on a bed. Any suitable material may be used for the substrate including absorbent paper, cellulose sheet or film, cardboard or the like. The beverage is then brought into contact with the chemical indicator.

In one arrangement the detector strip for the detection of gamma hydroxybutyrate may be in the form of an elongate bed with a small area impregnated with the chemical indicator material. In an alternative embodiment the detector strip may be absorbent paper impregnated with the chemical indicator material.

In one arrangement of this latter embodiment the detector strip comprises a strip of absorbent paper impregnated with ferrous chloride in a buffer. When this strip is brought into contact with a beverage containing gamma hydroxybutyrate the ferrous chloride is oxidised to ferric chloride by the presence of the gamma hydroxybutyrate.

In another embodiment the chemical will react to pH change. This is possible since gamma hydroxybutyrate is generally a clandestine product derived from butyrolactone solvent with caustic, and is left in mildly alkaline form. Its addition to beverages will change the alkality of the beverage. This is readily detectable using the proposed chemical formulation When there is typically 1 mg or more of gamma hydroxybutyrate in the beverage, the strip will turn blue when it comes into contact with the beverage.

This chemical spotting technology may also be used to provide a detection strip for use in the detection of amphetamines both pharmaceutical grade amphetamines and also those from ‘street drugs’ samples or clandestine tablets. In addition, it may be used to detect the presence of ketamines. Where a solution of Dragendorff reagent is impregnated in the absorbent material, it will produce a reddish colour when amphetamines are present or pink if ketamine is present. In addition, it will produce blackened speckles and tiny red/orange intense speckles when benzodiazepine is present. The blackened speckles are produced by the presence of bulking agents in the tablet and the red/orange intense speckles are due to the presence of the active benzodiazepine.

When aqueous solutions are applied to material impregnated with Dragendorff reagent blackening will occur although this blackening reduces where the solution is acidic. Thus in one arrangement in which Dragendorff reagent is used in the detection strip, an acidic salt may be incorporated in the paper to minimise this effect when the strip is brought into contact with beverages.

Alcohol may be detected by an enzyme-based reaction in which chemical changes occur, leading to a colour change.

The support to which the detection strips are releasably attached may be formed of any suitable materials. Suitable materials include those which are lightweight and have sufficient inherent strength not to become crushed in use and include cardboard and plastics material. The support may be formed from, for example, cardboard, and then coated with a plastics coating. The support material may be of any suitable configuration. Whilst it will generally be rectangular any shape may be used.

The detection strips may be attached to the support by any suitable means. In one arrangement at least a portion of the detection strip may be formed integrally with the support and may be connected thereto by a frangible flange. Thus in use the detection strip may be removed from the apparatus by breaking the detection strip from the support.

In an alternative arrangement, the detection strips may be attached to the support by means of an adhesive. Any suitable adhesive maybe used. In use the desired strip can be removed from the support by means of breaking the adhesive bond. In an alternative arrangement the detection strip may be removed from the support by breaking the strip above the point where it is attached to the support by means of the adhesive. For example where the detection strip is made of impregnated paper or has a cardboard base it may simply be torn from the support. However, where the detection strip has for example a plastics support, it may be provided with an area of weakness where breakage can occur. In an alternative arrangement, the adhesive may be a weak adhesive which will allow the detection strip to be removed from the support by, for example, peeling. Suitable adhesives include the repositional adhesive manufactured by 3M for use in Post-It™ notes.

The test apparatus of the present invention may be of any suitable configuration. In one arrangement, the strips may extend from the support in a finger-like arrangement. However, in an alternative arrangement, the strips will be laid across the face of the support. Where appropriate, the strips may be spaced in any suitable configuration on the support.

In a most preferred arrangement, the kit includes a cover for the support to protect the detection strips when they are not in use. The cover may be a wallet or box into which the support maybe placed. In an alternative arrangement, the cover is integral with the support. Thus in one arrangement the support maybe sized such that it maybe folded into two parts such that when “closed” a first part may be laid over the strips which are attached to the second part. In use, the first part will be folded back to reveal the strips.

In another arrangement, there may be a third part which at least partially folds over the first part when it is in a closed position to help hold it in position.

Thus in one particularly preferred arrangement, the kit of the present invention will be configured to resemble a match book. The overall size of the kit when closed maybe similar to a conventional match book or may be of the size of a business card.

Where the support is made from non-plastics material such as of cardboard, it maybe coated on one or both surfaces with a water-resistant coating to provide protection.

The support may also be printed with instructions for use and/or advice and/or guidance relating to drugs. Advertising material may also be present.

Thus a particular benefit of the present invention is that the user has a discrete kit providing them with a wide range of testing options and which is simple to use and operate.

Various modifications of the apparatus may be provided. Since it is often dark in venues, the apparatus may include means to enable the user to find the appropriate strip, such as raised portions on the stick shaped to correspond to the drug to be tested. Additionally or alternatively, fluorescent markings may be used or a small light device may be included.

The present invention will now be described, by way of example, with reference to the accompanying drawings:

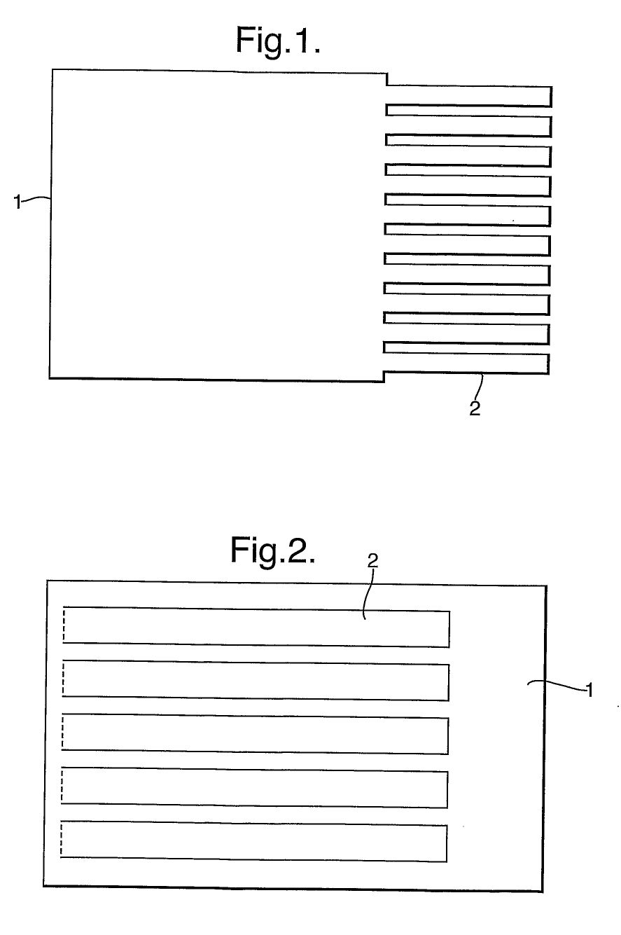

FIG. 1 illustrates one arrangement of the present invention;

FIG. 2 illustrates a second arrangement of the present invention;

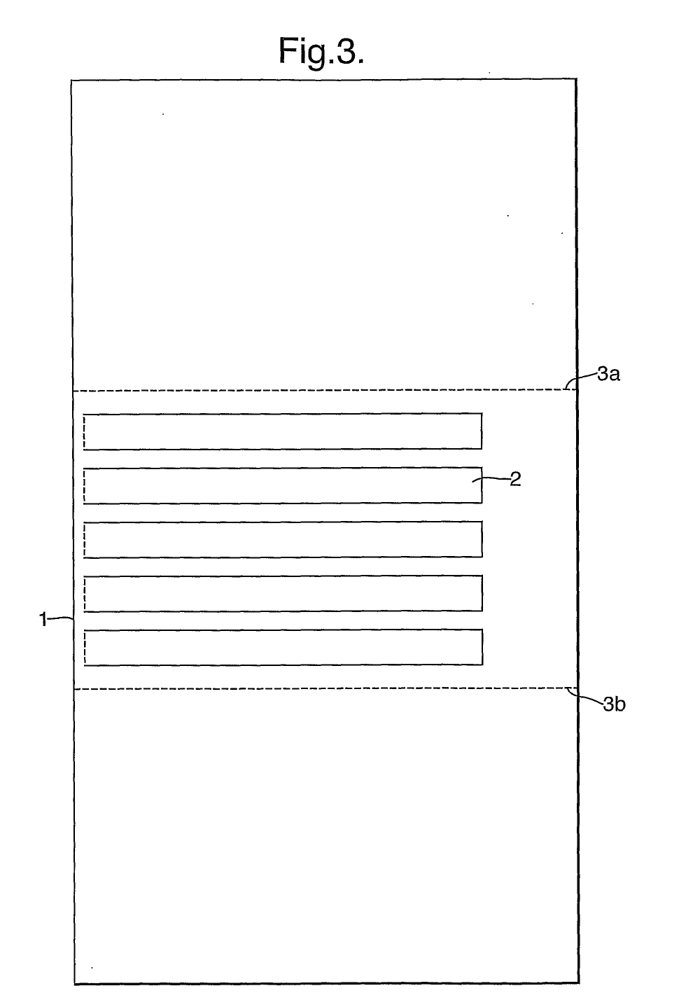

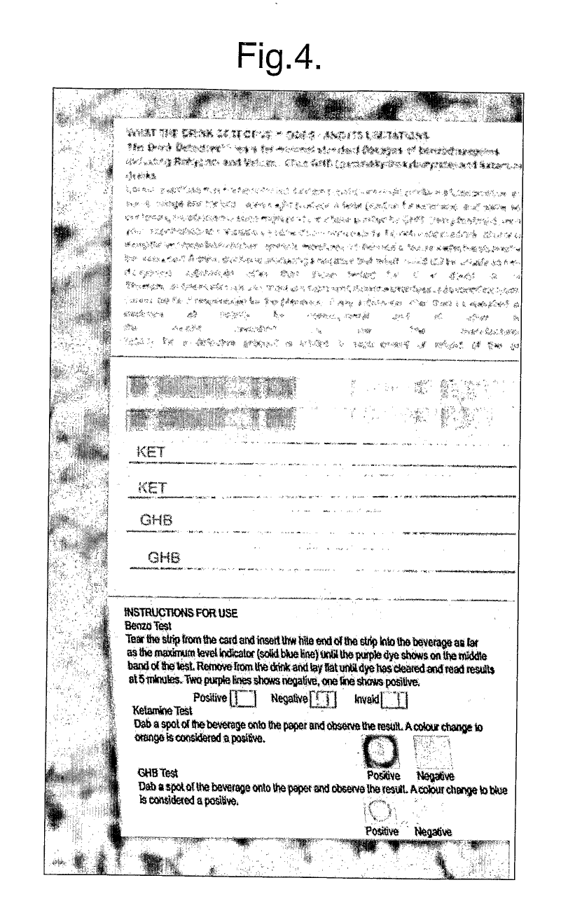

FIG. 3 illustrates a still further arrangement of the present invention;

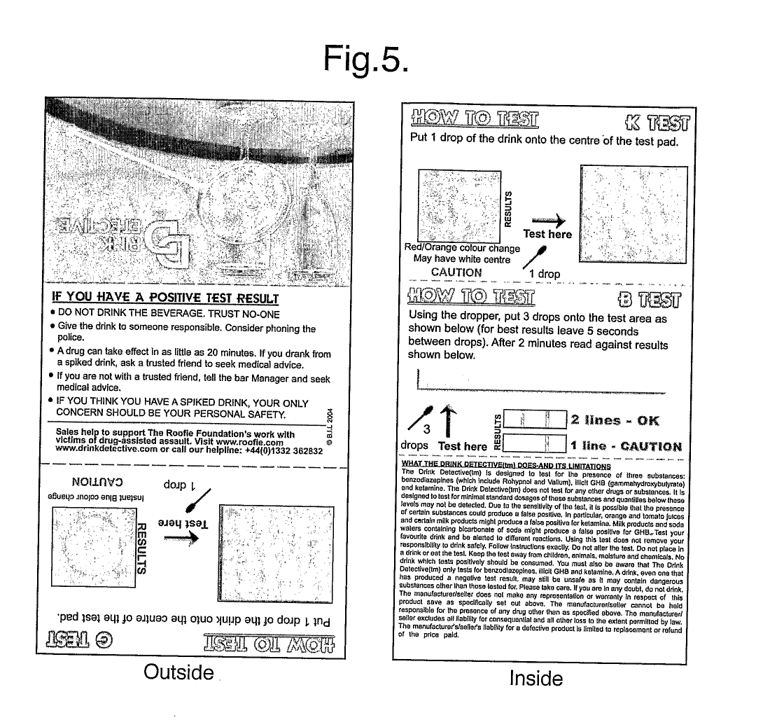

FIG. 4 illustrates a product of the present invention; and

FIG. 5 illustrates and alternative arrangement for a product of the present invention.

As illustrated in FIG. 1, the first arrangement of the present invention comprises a support 1 and a plurality of tear-off detection strips 2.

FIG. 2 illustrates a second arrangement where the detector strips 2 are attached by means of an adhesive to a face of the support 1.

FIG. 3 illustrates a preferred arrangement where the detector strips 2 are located on the face of a support 1. The support has folds 3a and 3b which enables it to be folded over the strips to protect the strips and to provide a match-book like configuration. An example of the product is shown in FIG. 4.

As illustrated in FIG. 5 each detector strip may be accompanied by instructions for that test. In this embodiment the liquid is applied to the strip rather than the strip being dipped into the drink. The strips in this configuration are longitudinal for the test for benzodiazepines and rectangular for the tests for gammahydroxybutyrate and ketamines.

1. A kit for detecting the presence of one or more drugs in a beverage comprising a support having a plurality of drug detector strips attached thereto.

2. The kit of claim 1 wherein the kit comprises detector strips for detecting at least two different drugs.

3. The kit of claim 2 where the kit comprises at least one strip for detecting benzodiazepines and at least one detector strip for detecting gamma hydroxy butyrate.

4. The kit of claim 2 wherein the kit comprises at least one strip for detecting one or more of drugs selected from the group consisting of ketamines, 3,4-methylenedioxy-N-methylamphetamine, cocaine, barbiturates, opiates, tricyclic antidepressants, acetaminophen, propoxyphene, and phencyclidine.

5. The kit of claim 2 wherein the kit comprises at least one strip for detecting alcohol.

6. The kit of claim 1 wherein each detector strip can detect one drug or drug type.

7. The kit of claim 1 wherein each detector strip can detect at least two drugs or drug type.

8. The kit of claim 1 wherein the kit comprises at least two of each type of detector strips.

9. The kit of claim 1 wherein the detector strip comprises reagents for immunoassay, chemical spot assay, enzyme assay, or a combination thereof.

10. The kit of claim 9 wherein the detector strip uses colour change chemistry.

11. The kit of claim 9 wherein the detector strip comprises a bed supporting the reagents.

12. The kit of claim 1 wherein the detector strips are releasably attached to the support.

13. The kit of claim 12 wherein the support is formed from cardboard or plastics material.

14. The kit of claim 1 wherein the detector strips are formed integrally with the support and are connected thereto by a frangible flange.

15. The kit of claim 14 wherein the support is formed from cardboard or plastics material.

16. The kit of claim 1 wherein the detection strips are attached to the support by means of an adhesive.

17. The kit of claim 1 further comprising a cover for the support.

18. The kit of claim 17 wherein the cover is integral with the support.

19. The kit of claim 18 wherein the support is sized such that when it is folded into two parts, a first part is laid over the strips which are attached to the second part.

20. The kit of claim 19 wherein a third part is present which at least partially folds over the first part when it is in a closed position to help hold the first part in position.

21. The kit of claim 17 wherein the cover is a wallet or box into which the support is placed.

22. The kit according to claim 1 wherein the kit is configured to resemble a match book.

23. The kit of claim 1 wherein the support is made from non-plastics material and is coated on at least one surface with a water-resistant coating to provide protection.

24. The kit of claim 1 wherein the support is printed with instructions for use.

25. A kit for detecting the presence of one or more drugs in a beverage comprising:
a support and;
a plurality of drug detector strips for detecting at least two drugs or drug types selected from the group consisting of benzodiazepines, gamma hydroxy butyrate, ketamines, 3,4-methylenedioxy-N-methylamphetamine, cocaine, barbiturates, opiates, tricyclic antidepressants, acetaminophen, propoxyphene, phencyclidine, and alcohol; wherein the detector strips are attached to the support; the detector strips comprise reagents for immunoassay, chemical spot assay, enzyme assay, or a combination thereof; and the detector strip uses color chance chemistry to indicate the presence of a drug.
